(19) United States
(12) Patent Application Publication
(10) Pub. No.: US 2009/0255544 A1
(43) Pub. Date: Oct. 15, 2009

(54) DEVICES AND METHODS FOR THE ENDOLUMENAL TREATMENT OF OBESITY

(75) Inventor: John A. COX, San Clemente, CA (US)

Correspondence Address: LEVINE BAGADE HAN LLP, 2400 GENG ROAD, SUITE 120, PALO ALTO, CA, 94303, US

(73) Assignee: USGI MEDICAL, INC., SAN CLEMENTE, CA (US)

(21) Appl. No.: 12/409,335

(22) Filed: Mar. 23, 2009

Related U.S. Application Data
(60) Provisional application No. 61/038,487, filed on Mar. 21, 2008.

(51) Int. Cl. A61B 17/00

(52) U.S. Cl. 128/898

(57) ABSTRACT

Devices and methods for forming and securing tissue folds and elongated invaginations in stomach tissue are used as a treatment for obesity. In a first embodiment, a plurality of tissue folds is formed in the fundus region of the stomach. In a second embodiment, one or more elongated invaginations is formed in the body region of the stomach. In a third embodiment, a plurality of tissue folds is formed in the fundus region of the stomach and one or more elongated invaginations is formed in the body region of the stomach. Additional embodiments include various combinations of tissue folds, elongated invaginations, and other reconfigurations of stomach tissue.

### CROSS-REFERENCE TO RELATED APPLICATIONS

This application claims benefit of priority to U.S. Provisional Patent Application No. 61/038,487, filed on Mar. 21, 2008, the content of which is incorporated herein by reference in its entirety.

### BACKGROUND

The present disclosure pertains to devices and methods for the endolumenal treatment of obesity. More particularly, the present disclosure relates to devices and methods for endolumenally manipulating stomach tissue, forming and securing tissue folds, forming and securing tissue invaginations, altering stomach tissue configuration, restricting the ability of stomach tissue to distend, altering the function of nerves located in or near stomach tissue, and/or altering hormone production from cells associated with stomach tissue.

The National Institutes of Health (NIH) estimate that about two-thirds of adults—133.6 million people—in the U.S. are overweight or obese, while almost 5% of adults—15 million Americans—are considered extremely obese. Obese adults are at increased risk of type II diabetes, hypertension, stroke, certain cancers, and other dangerous conditions.

The NIH estimates that being overweight or obese leads to $117 billion in medical spending a year, with $61 billion in direct costs and $56 billion in indirect costs.

As obesity rates continue to rise, patients are increasingly seeking surgical weight loss options. Bariatric surgery aids weight loss by restricting food intake and, in some operations, altering the digestive process. The Roux-en-Y Gastric Bypass Procedure (RYGBP) is the most commonly performed bariatric procedure, estimated to account for approximately 65% of weight loss surgeries performed in the U.S.

A study from the Agency for Healthcare Research and Quality (AHRQ) found that the number of bariatric surgeries grew by 400 percent between 1998 and 2002. In 2007, an estimated 205,000 people with morbid obesity in the U.S. will have undergone bariatric surgery and these numbers are expected to grow. Only 1% of the clinically eligible population is currently being treated for morbid obesity through bariatric surgery.

A major retrospective study published in the New England Journal of Medicine showed that gastric bypass reduced the risk of death in extremely obese patients by over 40% by lowering the incidence of diabetes, coronary artery disease and cancer.

The Roux-en-Y gastric bypass procedure involves creating a small stomach pouch out of a portion of the stomach and attaching it directly to the jejunum, bypassing a large part of the stomach and duodenum. The stomach is made very small to restrict the amount of food that can be consumed. The opening between the stomach pouch and the small intestine (called the stoma) is also made very small to slow the passage of food from the stomach. These restrictions help the patient feel full and limit the amount of food that can be eaten. In addition, by altering the path of the intestines, consumed food bypasses the duodenum so fat absorption is substantially reduced.

The RYGB procedure is performed either laparoscopically or in an open surgery. Alternative procedures for obtaining some or all of the benefits of bariatric surgery without requiring an open surgical or laparoscopic procedure would be preferred.

### SUMMARY

In a first aspect, endolumenal treatment of obesity in a minimally invasive manner includes a number of methods and devices. The devices are introduced endolumenally (e.g., transorally, transanally, etc.) into the patient's body and into or around the gastrointestinal (“GI”) tract. Once the instruments are positioned within the stomach, tissue within the stomach is temporarily engaged or grasped and the engaged tissue is manipulated by a surgeon or practitioner from outside the patient's body.

In engaging, manipulating, and/or securing the tissue, various methods and devices may be implemented. For instance, tissue securement devices may be delivered and positioned via an endoscopic apparatus for contacting a tissue wall of the pouch lumen, creating one or more tissue folds, and deploying one or more tissue anchors through the tissue fold(s). The tissue anchor(s) may be disposed through the muscularis and/or serosa layers of the pouch lumen. An endoscopic access assembly having an elongate body, a steerable distal portion, and multiple lumens defined therethrough may be advanced into a pouch per-orally and through the esophagus. A tissue manipulation assembly positioned at the distal end of a tubular body may be passed through the endoscopic assembly for engaging and securing the tissue.

Utilizing one or more of the instruments, the endoscopic access device may be used to pass the flexible body therethrough and into the stomach where it may be used to engage tissue and form folds, invaginations, or other reconfigurations of tissue which are secured via expandable tissue anchors expelled from the tissue manipulation assembly. Any number of tissue folds and/or invaginations, i.e., one or more, may be created in a uniform pattern or randomly throughout the stomach interior such that the stomach volume is reduced, stomach tissue is inhibited from distention, and stomach nerve function and/or hormone production are altered.

In an embodiment, a delivery catheter is advanced through a patient's mouth and esophagus and into the patient's stomach, with the delivery catheter including a flexible tube having a needle at its distal end and with a first tissue anchor assembly being contained within the flexible tube of the delivery catheter. One or more instruments associated with the delivery catheter are used to form a first tissue fold in the tissue of the stomach fundus, the tissue fold including a serosa-to-serosa contact of tissue on the peritoneal surface of the stomach fundus. The needle of the delivery catheter is passed through the first tissue fold, and a first tissue anchor assembly is deployed from the delivery catheter through the first tissue fold to thereby secure the first tissue fold. A first plurality of additional tissue folds is also secured in the tissue of the stomach fundus. A first elongated invagination of tissue is then formed in the body region of the stomach extending generally from the fundus toward the antrum, with the first elongated invagination including a serosa-to-serosa contact of tissue on the peritoneal surface of the stomach body region. A plurality of tissue anchor assemblies from the delivery catheter is deployed through the first elongated invagination of tissue to thereby secure the tissue.

In some embodiments, the first elongated invagination is located substantially on the anterior wall of the stomach body region. In other embodiments, the first elongated invagination is located substantially on the lateral wall of the stomach body region. In still other embodiments, a second elongated invagination is formed in the body region.

In alternative embodiments, various combinations of tissue folds, tissue invaginations, and other tissue reconfigurations are formed and secured at selected regions of the fundus and body of the stomach. The tissue folds, invaginations, and other reconfigurations have the effects of reducing stomach volume, inhibiting distention of stomach tissue, more effectively and more quickly force food down to the antrum, and/or favorably altering the nerve function and/or hormone production of stomach tissue to thereby creating signals of satiety.

### BRIEF DESCRIPTION OF THE DRAWINGS

FIG. 1 is a schematic representation of a tissue anchor assembly.

FIG. 2 is a schematic representation of a tissue anchor assembly securing a tissue fold.

FIG. 3 is an exploded view of a tissue anchor assembly delivery device.

FIG. 4 is an exploded view of a needle deployment assembly.

FIGS. 5A and 5B are perspective views of embodiments of endolumenal access systems.

FIG. 6 is an illustration of an endolumenal access system and tissue anchor delivery device advanced endolumenally into a stomach.

FIGS. 7A through 7C are side views of a tissue manipulation assembly and helical tissue engagement instrument engaging stomach tissue.

FIGS. 8A through 8J are illustrations showing a progression of an endolumenal obesity treatment procedure.

FIG. 9 is a schematic illustration of a stomach.

FIG. 10 is a schematic illustration of a stomach having a plurality of tissue folds formed in the fundus region of the stomach.

FIG. 10A is a schematic illustration taken along line A-A of FIG. 10.

FIG. 11 is a schematic illustration of a stomach having an elongated invagination formed in the lateral body region of the stomach.

FIG. 11A is a schematic illustration taken along line A-A of FIG. 11.

FIG. 12 is a schematic illustration of the exterior of the stomach shown in FIG. 11.

FIG. 13 is a schematic illustration of the exterior of a stomach with an elongated invagination formed in the anterior body region of the stomach.

FIG. 13A is a schematic illustration taken along line A-A of FIG. 13.

FIG. 14 is a schematic illustration of a stomach having a plurality of tissue folds formed in the fundus region and an elongated invagination formed in the lateral body region of the stomach.

FIGS. 14A and 14B are schematic illustration taken along lines A-A and B-B, respectively, of FIG. 14.

FIGS. 15A and 15B are schematic illustrations of a stomach having a plurality of tissue folds formed in the fundus region and an elongated invagination formed in the anterior body region of the stomach.

### DETAILED DESCRIPTION

Endolumenal surgical methods and devices are described herein. In several embodiments, the methods entail performing surgery through a patient's mouth or other natural orifices, reducing or eliminating the need for external incisions into the body. Operating through the body's natural orifices offers promise for faster healing times, less scarring and less pain which could lead to reduced hospitalization and quicker recovery.

In several embodiments, the endolumenal surgical procedures are performed using devices that have been developed by USGI Medical, Inc. of San Clemente, Calif. Several endoscopic access devices are described, for example, in the following U.S. patent applications:

TABLE 1

| U.S. patent application Ser. No. | Filing Date |
|---|---|
| 10/346,709 | Jan. 15, 2003 |
| 10/458,060 | Jun. 9, 2003 |
| 10/797,485 | Mar. 9, 2004 |
| 11/129,513 | May 13, 2005 |
| 11/365,088 | Feb. 28, 2006 |
| 11/738,297 | Apr. 20, 2007 |
| 11/750,986 | May 18, 2007 |
| 12/061,591 | Apr. 2, 2008 |

Several tissue manipulation and tissue anchor delivery devices are described in the following U.S. patent applications:

TABLE 2

| U.S. patent application Ser. No. | Filing Date |
|---|---|
| 10/612,109 | Jul. 1, 2003 |
| 10/639,162 | Aug. 11, 2003 |
| 10/672,375 | Sep. 26, 2003 |
| 10/734,547 | Dec. 12, 2003 |
| 10/734,562 | Dec. 12, 2003 |
| 10/735,030 | Dec. 12, 2003 |
| 10/840,950 | May 7, 2004 |
| 10/955,245 | Sep. 29, 2004 |
| 11/070,863 | Mar. 1, 2005 |

Endolumenal tissue grasping devices are described in several of the U.S. patent applications listed above, and in the following U.S. patent applications:

TABLE 3

| U.S. patent application Ser. No. | Filing Date |
|---|---|
| 11/736,539 | Apr. 17, 2007 |
| 11/736,541 | Apr. 17, 2007 |

Tissue anchors are described in several of the U.S. patent applications listed above, and in the following U.S. patent applications:

TABLE 4

| U.S. patent application Ser. No. | Filing Date |
|---|---|
| 10/841,411 | May 7, 2004 |
| 11/404,423 | Apr. 14, 2006 |
| 11/773,933 | Jul. 5, 2007 |

Each of the foregoing patent applications is hereby incorporated by reference in its entirety.

### Tissue Anchors and Delivery Devices and Methods

Several embodiments of the endolumenal surgical procedures described herein include the steps of grasping gastrointestinal (e.g., stomach) tissue to form a tissue fold and deploying or implanting a fold retaining device (e.g., a tissue anchor assembly) that is used to maintain the fold. For simplicity, the discussion herein will describe tissue anchor assemblies holding tissue folds, with it being understood that other portions or sections of tissue that do not constitute tissue folds are suitably retained by the tissue anchor assemblies. The following sections include descriptions of several embodiments of devices that are suitable for performing these and other endolumenal surgical procedures.

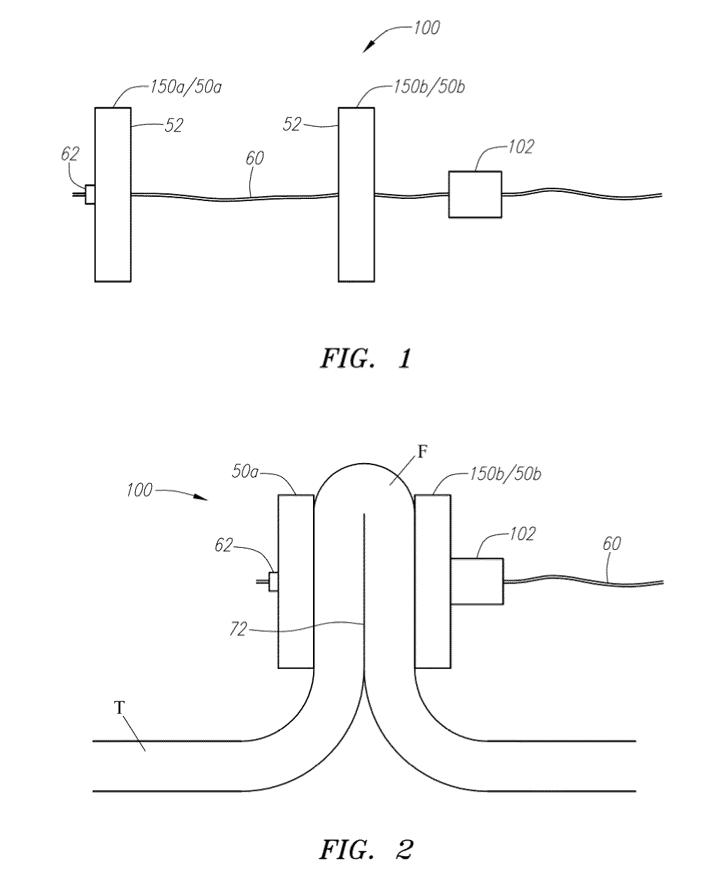

In several embodiments, a tissue anchor assembly is used to maintain a tissue fold in the gastrointestinal lumen. The preferred tissue anchor assemblies include tissue anchors such as those described in several of the U.S. patent applications incorporated by reference above, including Ser. Nos. 10/841,411, 11/404,423, and 11/773,933. A schematic representation of a suitable tissue anchor assembly is shown in FIG. 1.

Preferably, the tissue anchor assemblies include a pair of tissue anchors 150a/50a, 150b/50b slidably retained by a connecting member, such as a suture 60. A locking mechanism, such as a cinch 102, is also slidably retained on the suture 60. The cinch 102 is configured to provide a cinching force against the anchors 150a/50a, 150b/50b in order to impart a tension force on the suture. Accordingly, the tissue anchor assembly 100 is adapted to hold a fold of tissue, as shown in FIG. 2.

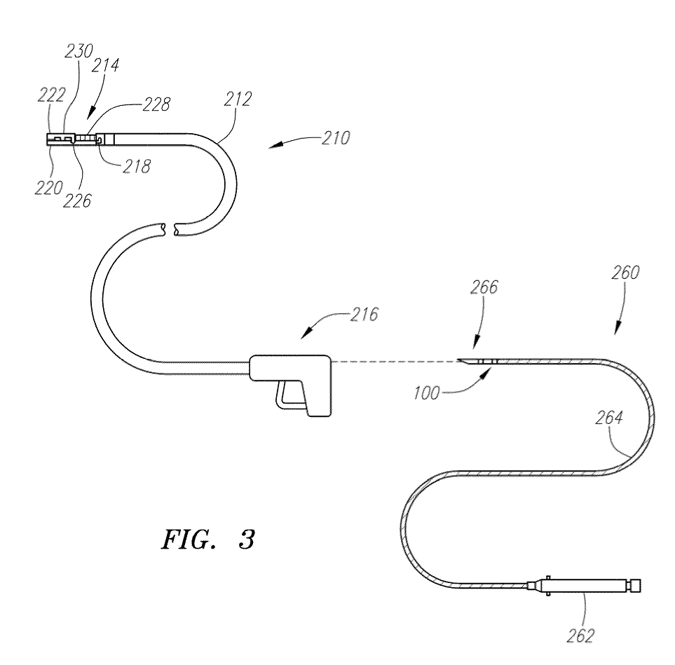

In several embodiments, a delivery device is used to deploy the tissue anchors and tissue anchor assemblies 100 endolumenally. An example of a suitable delivery device is shown in FIG. 3, and is described in more detail in U.S. patent application Ser. No. 11/070,846, which is hereby incorporated by reference in its entirety (including all references cited therein) as if fully set forth herein. The delivery device 208 is described briefly below.

In manipulating tissue or creating tissue folds, a device having a distal end effector may be advanced endolumenally, e.g., transorally, transgastrically, etc., into the patient's body, e.g., the stomach. The tissue may be engaged or grasped and the engaged tissue may be manipulated by a surgeon or practitioner from outside the patient's body. Examples of creating and forming tissue plications are described in further detail in U.S. patent application Ser. No. 10/955,245, filed Sep. 29, 2004, which is incorporated herein by reference, as well as U.S. patent application Ser. No. 10/735,030, filed Dec. 12, 2003, which is also incorporated herein by reference in its entirety.

In engaging, manipulating, and/or securing the tissue, various methods and devices may be implemented. For instance, tissue securement devices may be delivered and positioned via an endoscopic apparatus for contacting a tissue wall of the gastrointestinal lumen, creating one or more tissue folds, and deploying one or more tissue anchors through the tissue fold(s). The tissue anchor(s) may be disposed through the muscularis and/or serosa layers of the gastrointestinal lumen.

The delivery device 208 shown in FIG. 3 generally comprises a tissue manipulation assembly 210 and a needle deployment assembly 260. The tissue manipulation assembly 210 includes a flexible catheter or tubular body 212 which is configured to be sufficiently flexible for advancement into a body lumen, e.g., transorally, percutaneously, laparoscopically, etc. The tubular body 212 is configured to be torqueable through various methods, e.g., utilizing a braided tubular construction, such that when a handle 216 is manipulated and/or rotated by a practitioner from outside the patient's body, the longitudinal and/or torquing force is transmitted along the body 212 such that the distal end of the body 212 is advanced, withdrawn, or rotated in a corresponding manner.

A tissue manipulation end effector 214 is located at the distal end of the tubular body 212 and is generally used to contact and form tissue folds and/or to otherwise bring portions of tissue into apposition. The tissue manipulation end effector 214 is connected to the distal end of the tubular body 212 via a pivotable coupling 218. A lower jaw member 220 extends distally from the pivotable coupling 218 and an upper jaw member 222, in this example, is pivotably coupled to the lower jaw member 220 via a jaw pivot 226. The location of the jaw pivot 226 may be positioned at various locations along the lower jaw 220 depending upon a number of factors, e.g., the desired size of the “bite” or opening for accepting tissue between the jaw members, the amount of closing force between the jaw members, etc. One or both jaw members 220, 222 may also have a number of protrusions, projections, grasping teeth, textured surfaces, etc. on the surface or surfaces of the jaw members 220, 222 facing one another to facilitate the adherence of tissue between the jaw members 220, 222.

A launch tube 228 extends from the handle 216, through the tubular body 212, and distally from the end of the tubular body 212 where a distal end of the launch tube 228 is pivotally connected to the upper jaw member 222 at a launch tube pivot 230. A distal portion of the launch tube 228 may be pivoted into position within a channel or groove defined in upper jaw member 222, to facilitate a low-profile configuration of tissue manipulation end effector 214. When articulated, either via the launch tube 228 or other mechanism, the jaw members 220, 222 may be urged into an open configuration to receive tissue in the opening between the jaw members 220, 222.

The launch tube 228 may be advanced from its proximal end at the handle 216 such that the portion of the launch tube 228 that extends distally from the body 212 is forced to rotate at a hinge or pivot 230 and reconfigure itself such that the exposed portion forms a curved or arcuate shape that positions the launch tube opening perpendicularly relative to the upper jaw member 222. The launch tube 228, or at least the exposed portion of the launch tube 228, may be fabricated from a highly flexible material or it may be fabricated, e.g., from Nitinol tubing material which is adapted to flex, e.g., via circumferential slots, to permit bending.

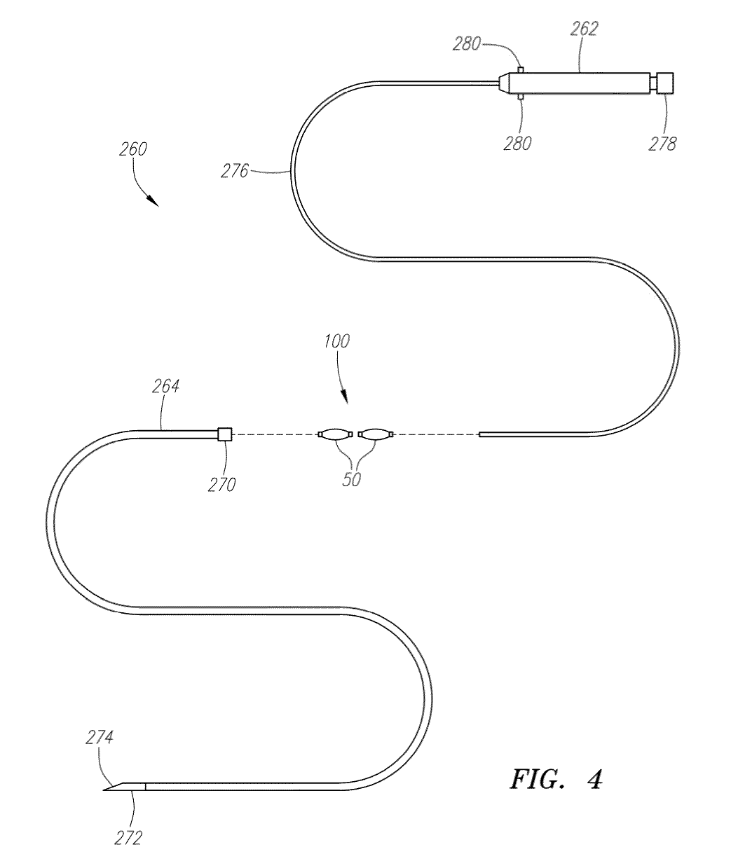

Once the tissue has been engaged between the jaw members 220, 222, a needle deployment assembly 260 is urged through the handle 216, though the tubular body 212, and out through the launch tube 228. The needle deployment assembly 260 may pass through the lower jaw member 220 via a needle assembly opening (not shown in the drawing) defined in the lower jaw member 220 to pierce through the grasped tissue. Once the needle deployment assembly has been passed through the engaged tissue, one or more tissue anchors of a tissue anchor assembly 100 (see FIG. 4) are deployed for securing the tissue, as described in further detail herein and in U.S. patent application Ser. No. 10/955,245, which has been incorporated by reference above.

FIG. 4 shows additional details relating to the needle deployment assembly 260. As mentioned above, a needle deployment assembly 260 may be deployed through the tissue manipulation assembly 210 by introducing needle deployment assembly 260 into the handle 216 and through the tubular body 212, as shown in the assembly view of FIG. 3, such that the needle assembly 266 is advanced from the launch tube and into or through approximated tissue. Once the needle assembly 266 has been advanced through the tissue, the anchor assembly 100 may be deployed or ejected. The anchor assembly 100 is normally positioned within the distal portion of a tubular sheath 264 that extends from a needle assembly control or housing 262. Once the anchor assembly 100 has been fully deployed from the sheath 264, the spent needle deployment assembly 260 may be removed from the tissue manipulation assembly 210 and another needle deployment assembly may be introduced without having to remove the tissue manipulation assembly 210 from the patient. The length of the sheath 264 is such that it may be passed entirely through the length of the tubular body 212 to enable the deployment of the needle assembly 266 into and/or through the tissue.

The elongate and flexible sheath or catheter 264 extends removably from the needle assembly control or housing 262. The sheath or catheter 264 and the housing 262 may be interconnected via an interlock 270 which may be adapted to allow for the securement as well as the rapid release of the sheath 264 from the housing 262 through any number of fastening methods, e.g., threaded connection, press-fit, releasable pin, etc. The needle body 272, which may be configured into any one of the variations described above, extends from the distal end of the sheath 264 while maintaining communication between the lumen of the sheath 264 and the needle opening 274.

An elongate pusher 276 comprises a flexible wire or hypotube that is translationally disposed within the sheath 264 and movably connected within tie housing 262. A proximally-located actuation member 278 is rotatably or otherwise connected to the housing 262 to selectively actuate the translational movement of the elongate pusher 276 relative to the sheath 264 for deploying the anchors from the needle opening 274. The tissue anchor assembly 100 is positioned distally of the elongate pusher 276 within the sheath 264 for deployment from the sheath 264. Needle assembly guides 280 protrude from the housing 262 for guidance through the locking mechanism described above.

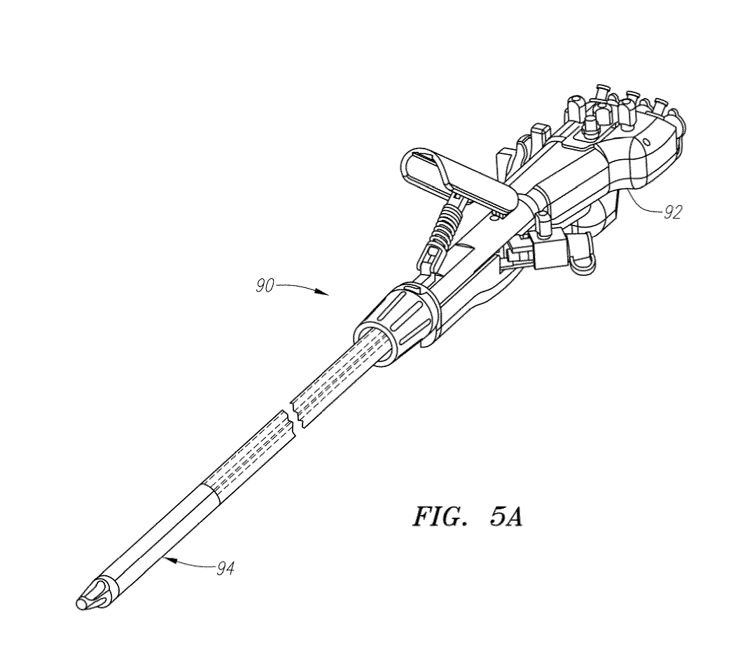

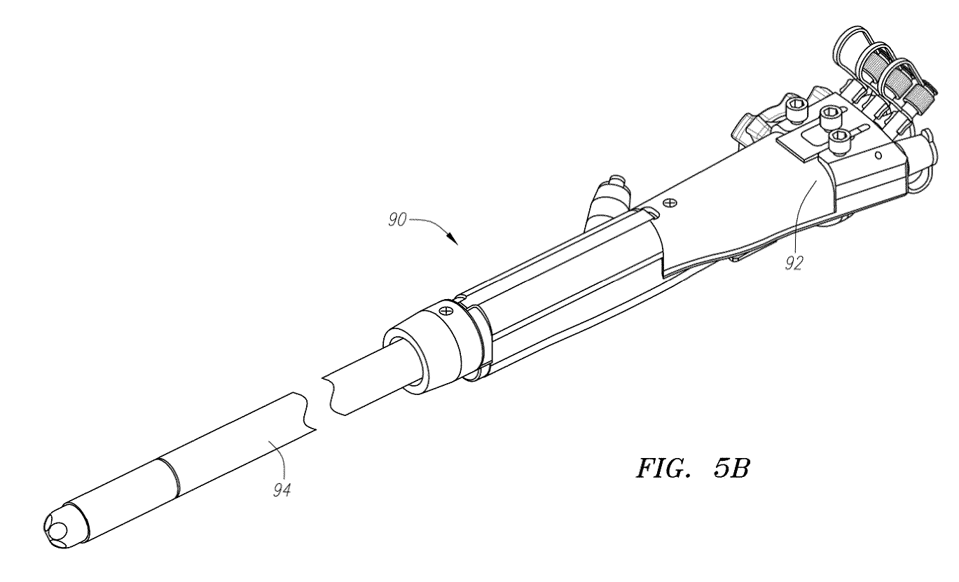

In several embodiments, the delivery device 210 and needle deployment assembly 260 are advanced into the gastrointestinal lumen using an endolumenal access system such as those described in the U.S. patent applications referenced above in Table 1. Two embodiments of endolumenal access systems are shown in FIGS. 5A and 5B.

The endolumenal access systems 90 illustrated in FIGS. 5A and 5B each include a handle 92 having control mechanisms for controlling device functions, and a multi-lumen, steerable overtube 94 having several features that are described more fully in U.S. patent application Ser. Nos. 11/750,986 and 12/061,591, which were incorporated by reference above.

### Endolumenal Surgical Treatment of Obesity

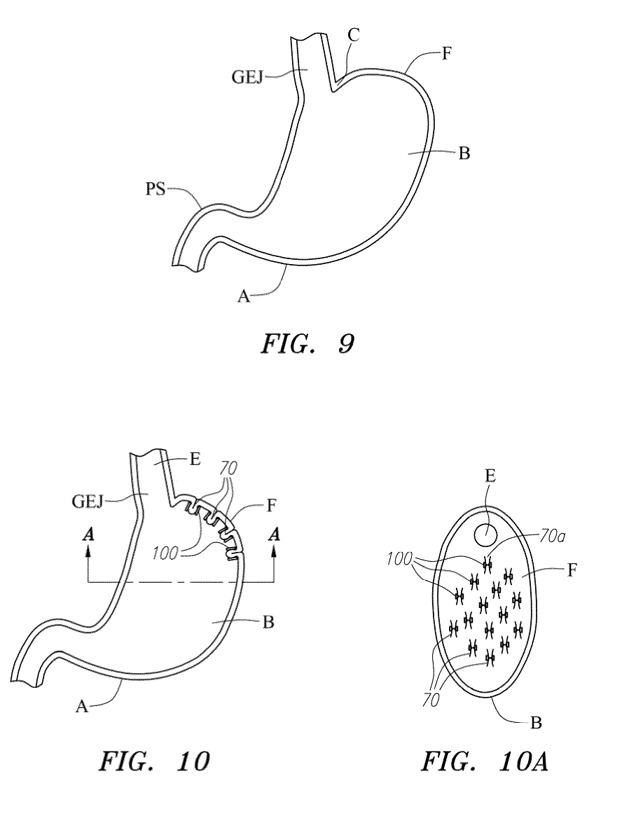

Referring to FIG. 9, the anatomy of the stomach can be divided into different segments on the basis of the mucosal cell types in relation to external anatomical boundaries. The cardiac segment C is immediately subjacent to the gastroesophageal junction GEJ and is a transition zone of the esophageal squamous epithelium into the gastric mucosa. The fundus F is the portion of the stomach that extends above the gastroesophageal junction. The body B or corpus of the stomach extends from the fundus F to the incisura angularis on the lesser curvature of the stomach. The majority of parietal acid forming cells are present in this segment. The fundus F and the body B function as the main reservoir of ingested food. The antrum A extends from the lower border of the body B to the pyloric sphincter PS. The majority of gastrin producing or G-cells are present in the antral mucosa. The main blood supply is variable but typically courses from the celiac axis into the gastric and gastroepiploic arcades. Nutrient vessels to the stomach course from the vascular arcades of the greater and lesser curvatures. These vessels penetrate the gastric wall in a perpendicular fashion and arborize horizontally in a dense vascular plexus throughout the wall of the stomach. For the most part, gastric innervation is provided by the vagus nerves which form a plexus around the esophagus and then reform into vagal trunks above the esophageal haitus. An extensive myenteric plexus is formed within the muscular wall of the stomach. Impulses from stretch or tension receptors within the gastric wall are transmitted to the nucleus tractus solitaris of the brain stem by afferent vagal fibers. These stretch/tension receptors within the fundus F and body B detect gastric distension or gastric pressure from ingested food.

The gastrointestinal lumen, including the stomach, includes four tissue layers, wherein the mucosa layer is the top tissue layer followed by connective tissue, the muscularis layer and the serosa layer. When stapling or suturing from the peritoneal side of the GI tract, it is easier to gain access to the serosal layer. In endolumenal approaches to surgery, the mucosa layers are visualized, and the muscularis and serosal layers are difficult to access because they are only loosely adhered to the mucosal layer. In order to create a durable tissue fold or other approximation with suture or staples or some form of anchor, it is important to create a serosa to serosa approximation. This is because the mucosa and connective tissue layers typically do not heal together in a way that can sustain the tensile loads imposed by normal movement of the stomach wall during ingestion and processing of food. In particular, folding the serosal layers in a way that they will heal together will form a durable tissue fold, plication, or elongated invagination. This problem of capturing the muscularis or serosa layers becomes particularly acute where it is desired to place an anchor or other apparatus transesophageally rather than intraoperatively, since care must be taken in piercing the tough stomach wall not to inadvertently puncture adjacent tissue or organs.

To treat obesity in a minimally invasive manner, a tissue manipulation and/or securement instrument is introduced per-orally through the patient's esophagus and into the stomach to perform a number of procedures. Alternatively, the instrument may be introduced transgastrically, percutaneously, etc., into the patient's body and into or around the stomach. Once the instrument is positioned within or adjacent to the stomach, tissue within or from the stomach is temporarily engaged or grasped and the engaged tissue is manipulated by a surgeon or practitioner from outside the patient's body. Examples of creating and forming tissue plications are described in further detail in U.S. patent application Ser. No. 10/955,245 filed Sep. 29, 2004 as well as in U.S. patent application Ser. No. 10/735,030 filed Dec. 12, 2003, each of which is incorporated herein by reference in its entirety.

Various methods and devices are implemented to engage, manipulate, and/or secure the tissue. For instance, in some embodiments, tissue securement devices are delivered and positioned via an endoscopic apparatus for contacting a tissue wall of the gastrointestinal lumen, creating one or more tissue folds, and deploying one or more tissue anchors through the tissue fold(s). The tissue anchor(s) are disposed through the muscularis and/or serosa layers of the tissue. When manipulating and securing tissue within a patient's body, a separate elongate shaft having a tissue engager on or near the distal end of the shaft may be utilized in conjunction with a tissue manipulation assembly. Such an instrument is generally utilized in endolumenal procedures where the tools are delivered through an endoscopic device.

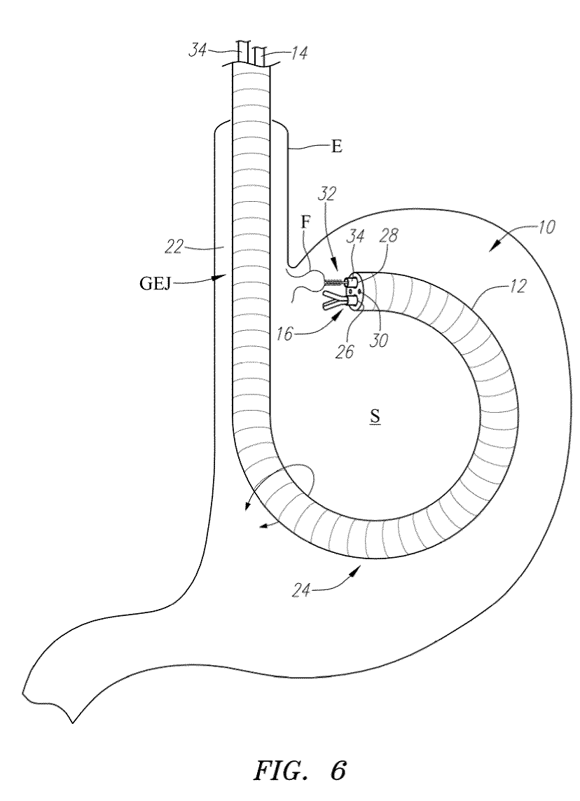

As illustrated in FIG. 6, one such example is shown in which an endoscopic assembly 10 is advanced into a patient's stomach S per-orally and through the esophagus E. The endoscopic assembly 10 includes an endoscopic device having a distal portion that is articulated and steered to position its distal end at a desired location within the stomach S. In some embodiments, the endoscopic assembly 10 is a flexible, steerable tube having a plurality of lumens extending therethrough. In other embodiments, the endoscopic assembly 10 is a flexible, steerable tube having one or more lumens extending therethrough, and also having the capability of shape-locking and/or rigidizing. In the latter embodiments, once located and configured, the assembly 10 may then be locked or rigidized to maintain its shape or configuration to allow for procedures to be performed on the tissue utilizing any number of tools delivered through the assembly 10. Shape-lockable or rigidizable assemblies 10 and variations thereof are described in further detail in U.S. patent application Ser. No. 10/346,709 filed Jan. 15, 2003, U.S. patent application Ser. No. 10/734,562 filed Dec. 12, 2003, U.S. patent application Ser. No. 11/750,986 filed May 18, 2007, and others of the applications that are incorporated by reference above.

The endoscopic assembly 10 generally comprises an endoscopic body 12 having an articulatable distal portion 24. The endoscopic body 12 may define at least first and second lumens 26, 28, respectively, through the endoscopic body 12 through which one or more tools may be deployed into the stomach S. Additional lumens may be provided through the endoscopic body 12, such as a visualization lumen 30, through which an endoscope may be positioned to provide visualization of the region of tissue. Alternatively, an imager such as a CCD imager or optical fibers may be provided in lumen 30 to provide visualization. An optional thin wall sheath may be disposed through the patient's mouth, esophagus E, and possibly past the gastroesophageal junction GEJ into the stomach S. The endoscopic body 12, having a covering 22 thereon, may be advanced through the esophagus E and into the stomach S while disposed in a flexible state.

The distal steerable portion 24 of the endoscopic body 12 is then articulated to an orientation, e.g., whereby the distal portion 24 facilitates engagement of tissue near and/or inferior to the patient's gastroesophageal junction GEJ. Accordingly, the distal steerable portion 24 may comprise a number of steering features, as described in further detail in U.S. patent application Ser. Nos. 10/346,709, 10/734,562, and 11/750,986, incorporated above. In those embodiments having shape-locking or rigidizing capabilities, with the distal steerable portion 24 disposed in a desired configuration or orientation, the endoscopic body 12 may be reversibly shape-locked to a rigid state such that the endoscopic body 12 maintains its position within the stomach S. Various methods and apparatus for rigidizing endoscopic body 12 along its length are also described in further detail in U.S. patent application Ser. Nos. 10/346,709, 10/734,562, and 10/346,709, incorporated above.

FIG. 6 further shows a tissue manipulation assembly 16 having been advanced through the first lumen 26 and a helical tissue engagement member 32 positioned upon a flexible shaft 34 advanced through the second lumen 28. The tissue wall of a body lumen, such as the stomach, typically comprises an inner mucosal layer, connective tissue, the muscularis layer and the serosa layer. To obtain a durable purchase, e.g., in performing a stomach reduction procedure, the helical tissue engagement member 32 is advanced into contact with the tissue and preferably engages the tissue F such that when the tissue engagement member 32 is pulled proximally to draw the engaged tissue F between the jaw members 18, 20 of tissue manipulation assembly 16, at least the muscularis tissue layer and the serosa layer is drawn into the tissue manipulation assembly 16. As the tissue manipulation assembly 16 may be utilized to grasp and secure the engaged tissue, any number of tools may be utilized with the tissue manipulation assembly 16, e.g., through the endoscopic body 12, to engage and manipulate the tissue of interest relative to the tissue manipulation assembly 16. For example, the tissue grasping devices described in U.S. patent application Ser. Nos. 11/736,539 and 11/736,541, incorporated above, are suitable for engaging and manipulating tissue in order to draw a tissue fold between the jaw members 18, 20 of the tissue manipulation assembly 16. For simplicity, the embodiments described below will include descriptions of methods using the helical tissue engagement member 32, with it being understood that other engagement devices are also suitable.

An illustrative example of a tissue manipulation instrument which may be utilized for endolumenally accessing tissue is described in further detail in U.S. patent application Ser. No. 11/070,863 filed Mar. 1, 2005 (US Pat. Pub. 2005/0251166 A1), which is incorporated herein by reference in its entirety. Such an instrument assembly generally comprises a flexible catheter or tubular body 14 which may be configured to be sufficiently flexible for advancement into a body lumen, e.g., transorally, percutaneously, laparoscopically, etc. Tubular body 14 may be configured to be torqueable through various methods, e.g., utilizing a braided tubular construction, such that when a proximally-located handle is manipulated and/or rotated by a practitioner from outside the patient's body, the longitudinal and/or torquing force is transmitted along body 14 such that the distal end of body 14 is advanced, withdrawn, or rotated in a corresponding manner.

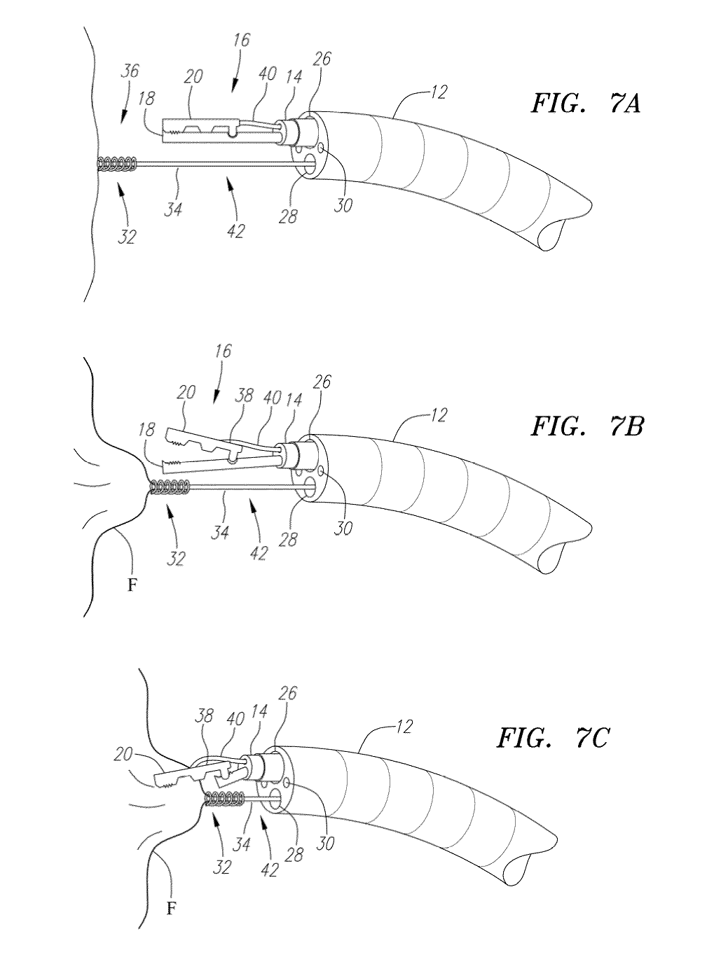

As shown in FIGS. 7A through 7C, the tissue manipulation assembly 16 is located at the distal end of the tubular body 14 and is generally used to contact and form tissue folds, as described above. The tissue manipulation assembly 16 may be connected to the distal end of the tubular body 14 via a pivotable coupling. The lower jaw member 18 extends distally from the pivotable coupling and the upper jaw member 20, in this example, may be pivotably coupled to the lower jaw member 18 via a jaw pivot. The location of the jaw pivot may be positioned at various locations along the lower jaw 18 depending upon a number of factors, e.g., the desired size of the “bite” or opening for accepting tissue between the jaw members, the amount of closing force between the jaw members, etc. One or both jaw members 18, 20 may also have a number of protrusions, projections, grasping teeth, textured surfaces, etc., on the surface or surfaces of the jaw members 18, 20 facing one another to facilitate the adherence of tissue between the jaw members 18, 20.

The launch tube 40 may extend from the handle, through the tubular body 14, and distally from the end of the tubular body 14 where a distal end of the launch tube 40 is pivotally connected to the upper jaw member 20 at a launch tube pivot. A distal portion of the launch tube 40 may be pivoted into position within a channel or groove defined in the upper jaw member 20, to facilitate a low-profile configuration of the tissue manipulation assembly 16. When articulated, either via the launch tube 40 or other mechanism, as described further below, the jaw members 18, 20 may be urged into an open configuration to receive tissue in the jaw opening between the jaw members 18, 20.

The launch tube 40 may be advanced from its proximal end at the handle such that the portion of the launch tube 38 that extends distally from body 14 is forced to rotate at a hinge or pivot and reconfigure itself such that the exposed portion forms a curved or arcuate shape that positions the launch tube opening to a position that is substantially perpendicular relative to the upper jaw member 20. The launch tube 40, or at least the exposed portion of the launch tube 38, may be fabricated from a highly flexible material or it may be fabricated, e.g., from Nitinol tubing material which is adapted to flex, e.g., via circumferential slots, to permit bending.

FIGS. 7A to 7C further illustrate one method for articulating a tissue manipulation assembly into an opened and closed configuration. As shown in FIG. 7A, the assembly may be delivered into a patient while in a low-profile configuration 40, e.g., trans-orally, trans-anally, percutaneously, through an endoscope, an endoscopic device, directly, etc., and desirably positioned relative to a tissue region of interest 36. In embodiments in which the endoscopic body 12 is shape-lockable or rigidizable, the endoscopic body 12 may be rigidized to maintain its configuration within the patient body. Alternatively, it may be left in a flexible state during the procedure.

The tissue region of interest 36 as well as the procedure may be visualized through the visualization lumen 30 or a separate imager. In either case, the tissue manipulation assembly 16 and the tissue engagement member 32 may be advanced distally out from the endoscopic body 12 through their respective lumens 26, 28. The tissue engagement member 32 may be advanced into contact against the tissue surface, as shown in FIG. 7A, and then rotated via its proximal handle until the tissue is engaged. The engaged tissue F may be pulled proximally relative to the endoscopic body 12 and the tissue manipulation assembly 16 may be actuated via its proximally located handle into an open expanded jaw configuration for receiving the engaged tissue F, as shown in FIG. 7B.

Once desirably positioned, the launch tube 40 may be urged proximally via its proximal end at the handle. Because of the jaw assembly pivot and the relative positioning of the upper jaw 20 along the lower jaw member 18 and the launch tube pivot along upper jaw member 20, the proximal movement of the launch tube 40 may effectively articulate the upper jaw 20 into an expanded jaw configuration, as shown in FIG. 7B. Proximally urging the launch tube 40 may also urge the lower jaw member 18 to pivot and form an angle relative to a longitudinal axis of the tubular body 14. The opening of the upper jaw 20 relative to the lower jaw 18 creates a jaw opening for grasping, receiving, and/or manipulating tissue. Moreover, the tissue manipulation assembly may also include a stop located adjacent to the jaw assembly pivot or within the pivot itself.

Once the launch tube 40 has been urged proximally, it may be locked into place thus locking the jaw configuration as well. Moreover, having the launch tube 40 articulate the jaw members 18, 20 in this manner eliminates the need for a separate jaw articulation and/or locking mechanism. Once the tissue has been pulled or manipulated between the jaw members 18, 20, the launch tube 40 may be pushed distally to actuate the jaw members 18, 20 into a closed, grasping configuration, as shown in FIG. 7C, for engagement with the tissue. As the launch tube 40 is urged distally through the elongate body 12, the lower jaw member 18 may be maintained at an angle relative to the tissue to further facilitate manipulation of the grasped tissue.

Although the launch tube 40 may be fabricated from different materials having differing flexibilities, it may also be fabricated from a single material, as mentioned above, where the flexible portion 38 may be configured, e.g., by slotting, to allow for bending of the launch tube 40 in a plane to form a single curved or arcuate section while the proximal rigid section may extend at least partially into the tubular body 14 to provide column strength to the launch tube 40 while it is urged distally upon the upper jaw member 20 and upon any tissue engaged thereby, as seen in the FIG. 7C.

Once the tissue has been engaged between the jaw members 18, 20, a needle assembly may be urged through the handle and out through the launch tube 40. The needle assembly may pass through the lower jaw member 18 via a needle assembly opening defined in the lower jaw member 18 to pierce through the grasped tissue. Once the needle assembly has been passed through the engaged tissue, one or more tissue anchors may be deployed for securing the tissue, as described in further detail in U.S. patent application Ser. No. 10/955,245, which has been incorporated by reference above.

The tissue engagement member 32 may be retracted from the tissue F or it may be left within the tissue while the tissue manipulation assembly engages and secures the tissue F. The tissue engagement member 32 is shown as a tissue piercing helix or corkscrew structure upon a flexible shaft 34. The tissue engagement member 32 may be rotated about its longitudinal axis to engage the tissue of interest by rotating its handle located on the proximal end of the flexible shaft 34.

A distal portion of the shaft 34 proximal to the engagement member 32 (or the entire length or a majority of the length of the shaft 34 in other variations) may include a marked section 42, as shown in FIGS. 7A to 7C. The tissue engagement member 32 and the flexible shaft 34 are rotated about its longitudinal axis to advance the engagement member 32 into the tissue region of interest 36. Accordingly, the marked section 42 may comprise any number of markings, designs, patterns, projections, textures, etc., which acts to provide a visual indication to the user as to the translational movement, rotation, direction of rotation, etc., of the engagement member 32 and the shaft 34 relative to the tissue region 36 when viewed from outside the patient body laparoscopically or endolumenally, for instance, through the visual lumen 30.

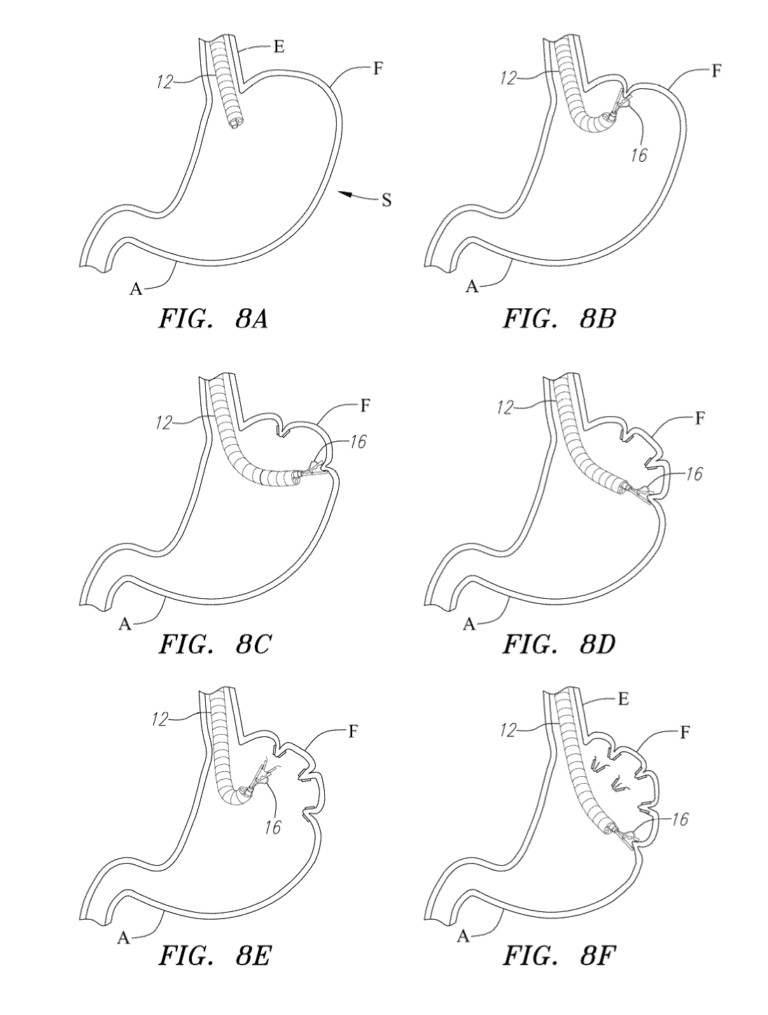

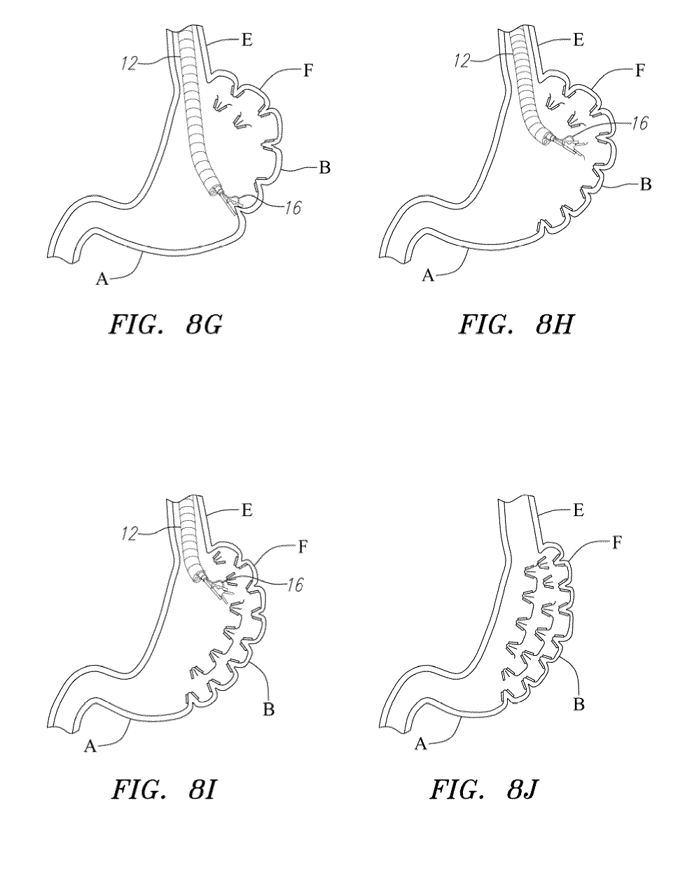

Utilizing the instruments described above, various endolumenal obesity-related procedures may be performed. For example, FIGS. 8A-J show a process view of an obesity treatment procedure being performed on a stomach. The remaining anatomy, including portions of the stomach S, has been omitted only for clarity. The flexible body 14 and tissue manipulation assembly 16, described above, are advanced per-orally, through the esophagus E, and into the stomach. The endoscopic body 12 may be used to pass the flexible body 14 therethrough and into the stomach, as shown in the figure. In an alternative method (and for methods described below), the endoscopic body 12 may be omitted entirely and the flexible body 14 and tissue manipulation assembly 16 are passed directly into the stomach.

Turning to the series of FIGS. 8A-J, a tissue manipulation assembly 16 is inserted per-orally into a patient's stomach S in an obesity treatment procedure. (FIG. 8A). The tissue manipulation assembly 16 forms a tissue fold and secures the fold with a tissue anchor assembly. (FIG. 8B). The tissue manipulation assembly 16 forms and secures additional tissue folds that are substantially aligned in a first row extending through a portion of the fundus F. (FIGS. 8C-D). The tissue manipulation assembly 16 forms and secures additional tissue folds in a random pattern or in rows or other patterns in the fundus F, thereby reducing stomach volume and/or reducing the ability of the fundus F to stretch to accommodate food. (FIGS. 8E-F). The tissue manipulation assembly 16 forms and secures additional tissue folds in random patterns or rows (vertical, horizontal, or diagonal) on the mid-body B anterior and lateral inner walls of the stomach. (FIGS. 8G-H). The tissue manipulation assembly 16 forms and secures additional tissue folds in random patterns or rows extending from the posterior wall of the fundus F into the mid-body B posterior inner wall of the stomach. (FIGS. 8I-J). The antral region A is not substantially altered.

Once within the stomach, the tissue manipulation assembly 16 is used to create approximated folds of tissue that are secured via expandable tissue anchors 52 expelled from the tissue manipulation assembly 16, as described above. A plurality of tissue folds 50, i.e., one or more, are created in a desired pattern or randomly throughout the stomach or other portions of the gastrointestinal lumen. In several embodiments, the locations of the tissue folds are selected to provide desired results. For example, tissue folds formed in the region of the fundus F have the effects of immobilizing the fundus, reducing the amount of distension that occurs to thereby prevent the fundus from accommodating the influx of food, and/or inducing satiety. As an additional example, tissue folds formed on the anterior wall of the stomach near the location of the vagal nerve branch (anterior, major) have the effect of compressing the wall and changing the effectiveness of the nerve branch, thereby inducing satiety and/or loss of appetite. As a still further example, tissue folds formed in the mid-stomach region create a bumpy Magenstrasse-like effect—i.e., a “central road” or narrow path constituting a gastric canal through which food that enters the stomach S through the esophagus E is quickly passed through the stomach to the antrum A and out of the stomach through the pylorus. Still further, tissue folds formed in one or a plurality of regions of the stomach will have the effect of reducing stomach volume, thereby preventing the stomach from accommodating the influx of food and inducing satiety and/or loss of appetite. In still further examples, tissue folds formed in multiple regions of the stomach will provide combinations of the foregoing results.

FIGS. 8A-J illustrate an example of an endolumenal obesity treatment method that includes forming and securing a plurality of tissue folds that are substantially aligned in rows extending along the inner wall of the fundus F and the mid-body B of the stomach. In the embodiment shown, three rows of tissue folds are formed extending from the fundus F through the mid-body B of the stomach, e.g., posterior, lateral, and anterior. The number of tissue folds included within each row will vary according to the size of the stomach and other anatomical factors, but will usually include from 1 to about 10 or more tissue folds, and preferably from about 2 to about 5 tissue folds.

In other embodiments, tissue folds are formed and secured in other parts of the stomach, instead of or in addition to the plurality of rows of tissue folds shown in the embodiment illustrated in FIGS. 8A-J. For example, as discussed above, in some embodiments, in addition to the rows of tissue folds shown in the embodiment illustrated in FIGS. 8A-J, another plurality of tissue folds are formed and secured in the fundus F region of the stomach. In other embodiments, tissue folds are formed on the anterior wall of the stomach near the location of the vagal nerve branch (anterior, major). In other embodiments, various combinations of the foregoing tissue fold distributions are created. In still other embodiments, a plurality of tissue folds are formed in regular patterns (e.g., substantially aligned, zig-zag, circular, serpentine, or other geometric pattern) or random patterns distributed throughout one or more areas of the stomach (e.g., fundus, mid-body, antrum, greater curvature) or throughout multiple areas of the stomach.

FIGS. 10 through 15 illustrate several additional embodiments of methods for the endolumenal treatment of obesity. In each of the illustrated embodiments, the tissue folds and elongated invaginations are formed in the stomach tissue endolumenally using the flexible body 14 and tissue manipulation assembly 16 described above, or other suitable medical instruments. In several embodiments, the flexible body 14 and tissue manipulation assembly 16 are advanced per-orally, through the esophagus E, and into the stomach. In some of the embodiments, the endoscopic body 12 is used to pass the flexible body 14 therethrough and into the stomach. In alternative methods, the endoscopic body 12 is omitted entirely and the flexible body 14 and tissue manipulation assembly 16 are passed directly into the stomach.

In the examples illustrated in the Figures, a tissue fold 70 generally includes a portion of tissue in which at least the muscularis layer is raised relative to its immediately surrounding regions of tissue, and in some cases in which a serosa-to-serosa contact 72 (see FIG. 2) is formed on the external (peritoneal) surface of the stomach, such that the muscularis and/or serosal layers, once secured, will reform and/or heal together to form a durable, reconfigured region of tissue. A tissue fold 70 is typically secured using a single fastener, such as a staple, suture, tissue anchor assembly 100, or other device. In the examples, an elongated invagination 80 generally includes an elongated folded region of tissue in which at least the muscularis layer is raised relative to its immediately surrounding regions of tissue, and in some cases in which an elongated serosa-to-serosa line of contact 82 is formed on the external (peritoneal) surface of the stomach and a region of tissue 84 is substantially enclosed within the reconfigured stomach volume defined in part by the line of contact 82, such that the muscularis and/or serosal layers, once secured, will reform and/or heal together to form a durable, reconfigured region of tissue. An elongated invagination 80 is typically secured using a plurality of fasteners, substantially each adjacent pair of which is spaced apart by a distance no greater than that which will result in reformation of at least a substantial portion of the tissue region extending between the adjacent pair of fasteners. An elongated invagination 80 will typically have a length (i.e., length of the line of contact 82) of from about 0.5 cm up to about 30 cm or more, depending upon the size and shape of the stomach.

In the embodiment shown in FIGS. 10 and 10A, a plurality of tissue folds 70 is formed in the fundus region F of the stomach. Each tissue fold 70 is secured by a tissue anchor assembly 100. The total number and locations of the tissue folds 70 that are formed and secured in the fundus region F will depend on the size and locations of the tissue folds and the size and shape of the fundus region F. Typically, a first tissue fold 70a is formed near the gastroesophageal junction GEJ and is secured with a tissue anchor assembly 100. Then, additional tissue folds 70 are formed and secured in the region of the fundus F moving from the GEJ toward the body B region of the stomach. In some embodiments, the additional tissue folds 70 are arranged in rows or other patterns aligned with the first tissue fold 70a. In other embodiments, the additional tissue folds 70 are arranged randomly throughout the fundus F.

As shown in FIGS. 10 and 10A, in comparison to the unaltered size and shape of the stomach illustrated in FIG. 9, the fundus region F after forming and securing the plurality of tissue folds 70 with tissue anchor assemblies 100 is substantially flattened and reduced in size. As noted previously, the secured tissue folds 70 substantially restrict and reduce the amount of distention that occurs to thereby prevent the fundus F from accommodating the influx of food, and/or inducing satiety. In addition, the less distendable fundus F will “tug” on the GEJ as the stomach is filled with food to create a sensation of fullness.

Moreover, it is believed that the alteration of the stomach tissue in the fundus region F alters the production of Ghrelin, a prehormone that is produced predominantly in epithelial cells lining the fundus. Ghrelin is an important factor in the regulation of energy, and functions by increasing hunger through its action on hypothalamic feeding centers. Ghrelin also appears to suppress fat utilization in adipose tissue. By forming and securing tissue folds in the fundus, the fundus tissue is disrupted and heals in thickened ridges or pinches. The modified tissue will not produce Ghrelin at a normal rate, which has the effect of reducing a patient's feeling of hunger.

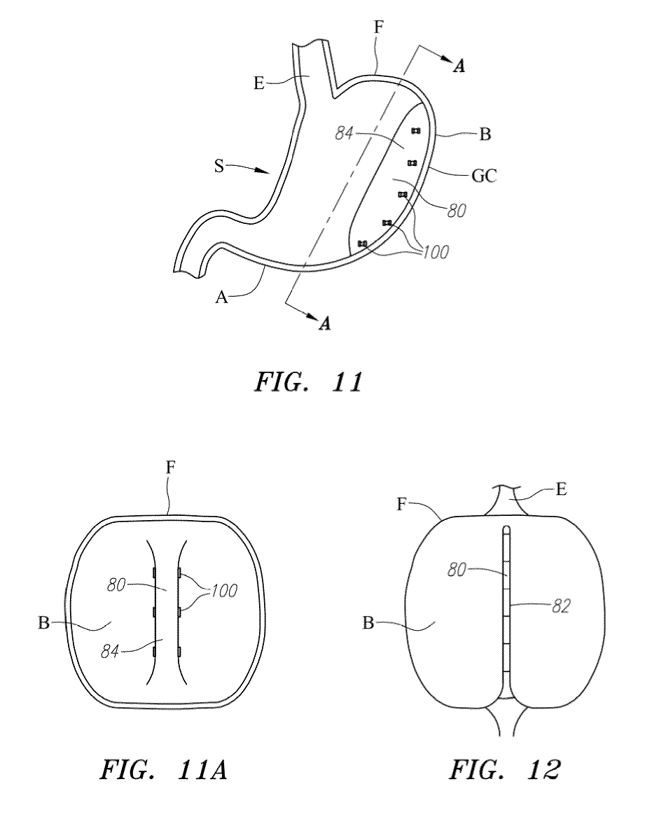

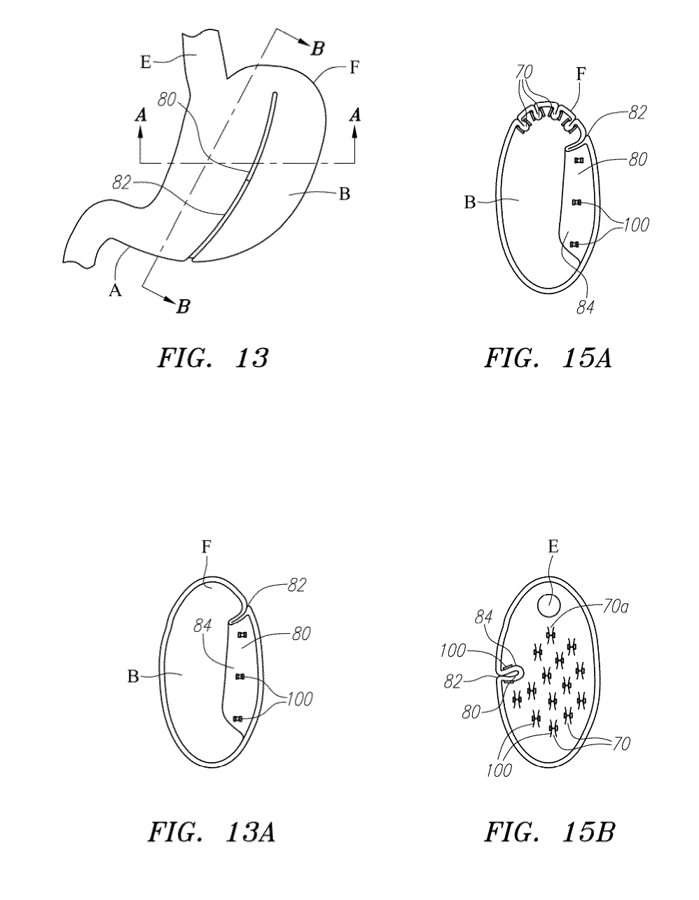

Turning next to FIGS. 11, 11A, and 12, there is shown a stomach S in which an elongated invagination 80 has been endolumenally formed and secured using a plurality of tissue anchor assemblies 100. In the embodiment shown, the elongated invagination 80 is a substantially continuous tissue fold extending longitudinally over the lateral body B of the stomach S from approximately the fundus F through the majority of the greater curvature GC. In another embodiment, illustrated in FIGS. 13 and 13A, the elongated invagination 80 is a substantially continuous tissue fold extending longitudinally over the anterior body B of the stomach S from approximately the fundus F through the majority of the greater curvature GC. In still other embodiments, the elongated invagination 80 is discontinuous, or is relatively shorter in length. In still other embodiments, multiple longitudinally-directed elongated invaginations 80 are formed in the posterior, lateral, and/or anterior wall of the body B of the stomach.

The elongated invaginations have the effect of reducing the effective volume of the stomach, and of shaping the stomach interior into a substantially tubular form. The tubular form of the stomach causes food to be forced down to the antrum A region more efficiently and quickly, thereby creating signals of satiety in the patient.

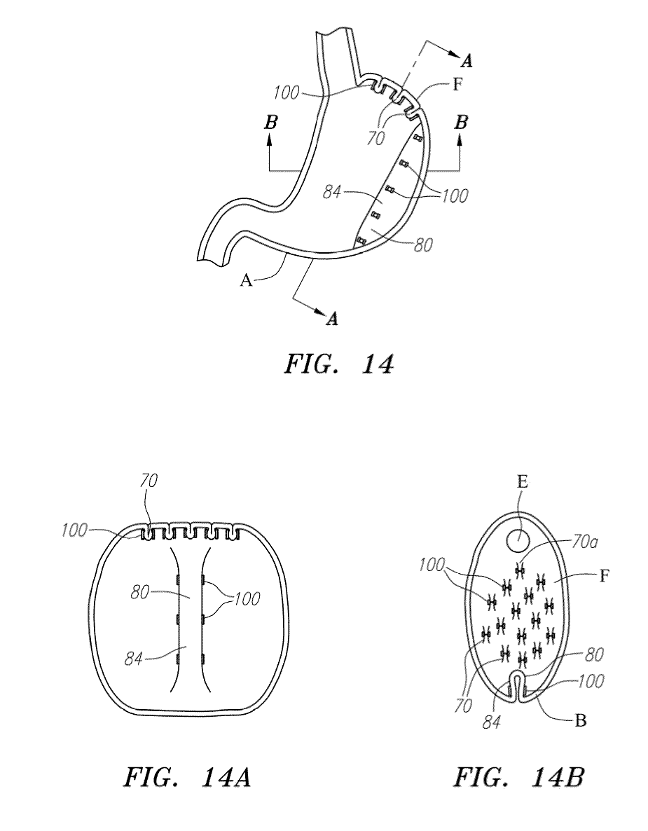

Turning next to FIGS. 14, 14A, and 14B, there is shown an embodiment of an endolumenal obesity treatment including a combination of tissue folds 70 formed in the fundus region F and a longitudinal elongated invagination 80 formed in the lateral wall of the body region B of the stomach. Each of the tissue folds 70 in the fundus region F is secured using a tissue anchor assembly 100. The elongated invagination 80 is secured by a plurality of tissue anchor assemblies 100. In another embodiment, shown in FIGS. 15A and 15B, the elongated invagination 80 is formed in the anterior wall of the body region B. In each embodiment, the tissue folds 70 and elongated invagination(s) 80 are formed endolumenally using instruments advanced through the esophagus E into the stomach S of the patient.

The combination of tissue folds 70 formed in the fundus region F and elongated invaginations 80 formed in the body region B provide a combination of the desirable effects described above for each of these stomach tissue alterations. Additional combinations of the therapeutic methods described herein will obtain similar results.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. An endolumenal treatment method, comprising:
advancing a delivery catheter through a patient's mouth and esophagus and into the patient's stomach, the delivery catheter including a flexible tube having a needle at its distal end, with a first tissue anchor assembly being contained within the flexible tube of the delivery catheter;
forming a first tissue fold in the tissue of the stomach fundus, the tissue fold including a serosa-to-serosa contact of tissue on the peritoneal surface of the stomach fundus;
passing the needle of the delivery catheter through the first tissue fold;
deploying the first tissue anchor assembly from the delivery catheter through the first tissue fold to thereby secure the first tissue fold;
securing a first plurality of additional tissue folds in the tissue of the stomach fundus;
forming a first elongated invagination of tissue in the body region of the stomach extending generally from the fundus toward the antrum, the first elongated invagination including a serosa-to-serosa contact of tissue on the peritoneal surface of the stomach body region; and
deploying a plurality of tissue anchor assemblies from the delivery catheter through the first elongated invagination of tissue to thereby secure the tissue.

2. The endolumenal treatment method of claim 1, wherein the first elongated invagination is located substantially on the anterior wall of the stomach body region.

3. The endolumenal treatment method of claim 1, wherein the first elongated invagination is located substantially on the lateral wall of the stomach body region.

4. The endolumenal treatment method of claim 1, further comprising the step of deploying a plurality of tissue anchor assemblies from the delivery catheter through a second elongated invagination of tissue formed in the body region of the stomach.

5. The endolumenal treatment method of claim 1, wherein the first elongated invagination has a length of from about 0.5 cm to about 30 cm.

6. The endolumenal treatment method of claim 1, further comprising the step of viewing the securing and deploying steps through an endoscope extending through a lumen of the delivery catheter.

7. The endolumenal treatment method of claim 1, further comprising the step of securing a second plurality of additional tissue folds in the tissue of the stomach body region.

8. The endolumenal treatment method of claim 7, wherein said second plurality of additional tissue folds are substantially aligned in one or more rows extending longitudinally from the fundus toward the antrum.

9. The endolumenal treatment method of claim 1, wherein the first elongated invagination comprises a substantially continuous line of contact of serosal tissue on the peritoneal surface of the stomach extending substantially from the fundus to the antrum.

10. The endolumenal treatment method of claim 4, wherein the first elongated invagination and second elongated invagination each comprise a substantially continuous line of contact of serosal tissue on the peritoneal surface of the stomach extending substantially from the fundus to the antrum.
